(19) United States
(12) Patent Application Publication
(10) Pub. No.: US 2016/0287661 A1
(43) Pub. Date: Oct. 6, 2016

(54) METHOD FOR TREATING CANCER BY ADMINISTERING ATP AND POLYMYXIN B

(71) Applicant: Universidad de Santiago de Chile, Santiago (CL)

(72) Inventors: Claudio Acuna, Santiago (CL); Claudio Capelli, Santiago (CL); Claudio Coddou, Santiago (CL); Alejandro Escobar, Santiago (CL); Monica Imarai, Santiago (CL); Yohana Labra, Santiago (CL); Ximena Lopez, Santiago (CL); Miguel Rios, Santiago (CL); Mercedes Lopez, Santiago (CL)

(21) Appl. No.: 15/013,320

(22) Filed: Feb. 2, 2016

Related U.S. Application Data
(62) Division of application No. 14/181,810, filed on Feb. 17, 2014, now abandoned.
Application No. 14/181,810 is a continuation of application No. PCT/CL2012/000043, filed on Aug. 17, 2012.

(30) Foreign Application Priority Data
Aug. 18, 2011 (CL) 2020-2011

(51) Int. Cl. A61K 38/12; C12N 5/0783; C12N 5/078; A61K 31/7076

(52) U.S. Cl. CPC A61K 38/12; A61K 31/7076; C12N 5/0637; C12N 5/0648; C12N 2501/998; C12N 2500/40

(57) ABSTRACT

A method for treating cancer. The method including administering to a subject in need thereof, a combination including an effective amount of polymyxin B in combination with an effective amount of ATP, wherein the combination is effective in treating a tumor or cancer in the subject.

### CROSS-REFERENCE TO RELATED APPLICATIONS

This is a divisional application of U.S. Ser. No. 14/181,810, files Feb. 17, 2014, which is a continuation of International Application No. PCT/CL2012/000043 filed on Aug. 17, 2012, which claims priority to Chilean Application No. 2020-2011 filed on Aug. 18, 2011, each of which are incorporated herein by reference in their entirety.

### TECHNICAL FIELD

The present invention is related to the use of polymyxin B in the depletion of CD4CD25 regulating T lymphocytes in vitro. Specifically, as coadjuvant in the inhibition of tumor proliferation in mice. More particularly, the use of polymyxin B and ATP in the treatment of cancer. The present invention is aimed to the use of Polymyxin B and ATP. More particularly, the current invention is addressed to the use of Polymyxin B and ATP in the treatment of cancer orientated to the depletion of CD4CD25 regulating T lymphocytes. This use is complementary to those of oncological immunotherapy applied to prostate, lung, bile, colon, kidney, pancreas, breast, ovarian, skin, recto, liver, encephalon, urinary bladder, stomach, endometrial, buco-pharyngeal, larynges, esophagus, cervical cancer.

### BACKGROUND ART

During the last decade an increase in the cancer incidence has been restricted at world level, stand by a significant problem in public health (see Redondo, P. (2000). Update on melanoma: incidence, development and biological aspects. An. Sist Sanit Navar. 23 (1): 67-92). In our country, cancer, represents the second cause of death population over 20 years, which means 17,266 deaths in a year (see Medina E., Kaempffer, A. (2000). Mortalidad del adulto en Chile. Rev. Méd. Chile. 128: 1144-49). Modern techniques of surgery and transplant, new or more effective drugs and better methods of irradiation have permitted treat with promissory results some of these pathologies (see Silverman, L. B., Weinstein, H. J. (1997). Treatment of Childhood Leukemia Curr. Opin. Oncol. 9: 2660-8. Review). Currently, it have been used further of the conventional therapies for the treatment of cancer, the immunotherapeutic approach, whose objective is stimulate the immune system to produce a specific response against the tumor through therapeutic vaccines or treatment, producing thus its elimination or reduction. Although there is evidence of that tumor cells are efficiently recognized by cell of the immune system after the vaccine treatment, many times this recognition is not enough to eliminate the pathology. This fact is due to different human cancer are able to actively inhibit the proper development of the immune response, through different mechanisms as the generation or inducing of CD4CD25 regulating T cells (Tregs) forward the tumor infiltrate inducing immune-suppression forward the effectors lymphocytes (see regulatory T cells in cancer, Beyer, 2006 and Identification of a New Subset of Myeloid Suppressor Cells, Filipazzi, 20074,5). Currently there are many clinical studies under advanced phases whose objective is modulating the immune response in patient having in mind that a immune response can be originated against tumor cells, these treatments are administered with other forms of “targeted therapy” as the therapies based on reinforcing the immune responses against cancer using modifiers of the biological response or inhibitors of the regulatory immune response. Among these last, several efforts have been developed with the aim of generating a therapy targeted by drugs or molecules which looking for depletion of this Tregs CD4CD25 population in human beings.

Currently there are two “targeted therapies” which are probed having a good efficacy in the process of depletion to this Tregs CD4CD25 population in human beings. The first contemplates the use of cyclophosphamide, which is used as a common drug in immunotherapy due to it has a cytotoxic effect aimed to tumor cells. Besides this property, recently it was discovered that when this drug is administered under low dose has a immunomodulating effect depleting Tregs without affecting T helper lymphocyte populations neither it is T cytotoxic (see Different mechanisms for anti-tumor effects of low- and high-dose cyclophosphamide. Motoyoshi. 2006). Another depletion mechanism of these regulating cells was developed by the generation of a specific drug named ONTAK® or Dinileukin Diftitox. This corresponds to a fusion protein between human IL-2 and the enzymatically active domains of the diphtheria toxin, which acts over cells presenting the receptor of high affinity to IL-2 (constituted by CD25, CD122 and CD132) in its cell membrane. Once ONTAK® interacts with the membrane, it is internalized by endocytosis and inhibits the protein synthesis, finally leading to the cellular apoptosis (see IL-2 immunotoxin denileukin diftitox reduces regulatory T cells and enhances vaccine-mediated T-cell immunity. Litzinger. 2007). To both cases higher doses can affect the effectors lymphocytes populations reducing also once the antitumor response.

Recently, it has been determined that Tregs CD4CD25 cells differentially express a molecule named P2X7 receptor (P2X7R) in a functional way as difference to other lymphocytes. P2X7R is member of P2X ATP-depending ion channels. One of the features of this receptor is that under low concentrations of ATP is induced a formation of pores, which have been associated to the induction of cellular death by apoptosis (see Aswad F, Kawamura H, Dennert G. High sensitivity of CD4+CD25+ regulatory T cells to extracellular metabolites nicotinamide adenine dinucleotide and ATP: a role for P2X7 receptors. J Immunol. 2005 Sep 1;175(5):3075-83). In this cellular types have been demonstrated that two no specific agonists of the P2X7 receptor, ATP and recently NAD, are able to induce the cellular death mediated by the activation of P2X7, either in vivo as in vitro (see Ferrari D, Pizzirani C, Adinolfi E, Forchap S, Sitta B, Turchet L, Falzoni S, Minelli M, Baricordi R, Di Virgilio F. The antibiotic polymyxin B modulates P2X7 receptor function. J. Immunol. 2004 Oct 1;173(7):4652-60 y F. Aswad, Kawamura H., Dennert, High sensitivity of CD4⁺CD25⁺ regulatory T cells to extracellular metabolites nicotinamide adenine dinucleotide an ATP: a role for P2X7 receptors, J. Immunol. 175 (2005), pp. 3075-3083). Under this context it has been described several cyclic and cationic peptides as gramicidine and polymyxin B, which are able to put sensitive the activation of the P2X7R receptor. These polypeptides per se are able to induce the production of interleukin 1 beta in macrophages y and dendritic cells deriving from mouse spinal cord, to levels which can be compared to the ones induced by ATP, an independent way the P2X7 receptor (see Allam R, Darisipudi M N, Rupanagudi K V, Lichtnekert J, Tschopp J, Anders H J. Cyclic Polypeptide and Aminoglycoside Antibiotics Trigger IL-1b Secretion by Activating the NLRP3 Inflammasome. J Immunol. 2011 Mar 1;186(5):2714-8). On the other hand, only the polymyxin B antibiotic (PMB), a cyclic and cationic peptide obtained from the bacteria Bacillus polymyxa, is able to be sensitive in the required way to lower ATP concentrations ATP to obtain the apoptotic effect (see Hubert S, Rissiek B, Klages K, Huehn J, Sparwasser T, Haag F, Koch-Nolte F, Boyer O, Seman M, Adriouch S. Extracellular NAD+ shapes the Foxp3+ regulatory T cell compartment through the ART2-P2X7 pathway. J Exp Med. 2010 Nov 22;207(12):2561-8. Epub 2010 Oct 25) in cell lines expressing P2X7R. Before, a similar effect had been described to the Mihich's working group, in cell lines, however, the mechanism had not been clarified. They determine that PMB induces cell death in eukaryote cell lines as the tumor cell of lymphoma EL4 y EL4/ADM, the tumor cell lines C1498 y REH (cells of acute myeloid leukemia) (see Verstovsek S, Maccubbin D L, Ehrke M J, Mihich E. Polymyxin B-mediated lysis of tumor cells. Int Arch Allergy Immunol. 1993;100(1):47-52). Gramicidine is a polypeptide antibiotic produced by the gram positive bacteria Bacillus brevi, acting the similar way to polymyxin as cationic detergent, altering the permeability of the bacterial cytoplasmatic membrane, which produces changes in the intracellular cation concentration, especially potassium. Gramicidine is inactive in serum and in body liquids and is efficacy only by topical way, further it is not used in systemic way due to a high toxicity over eukaryote cells.

The mechanism by which acts the polymyxin B is only clear in prokaryotes, in these the external membrane of the cell wall of Gram negative bacteria is composed mainly by lipopolysaccharide (LPS). LPS is constituted by a lipid motive named lipid A which is covalently linked with a polysaccharide. Lipid A is the responsible of the high endotoxic and immunogenic effect of LPS in mammals. Polymyxin B is a cationic peptide having a total charge equal to 15 and selectively acts over Gran positive bacteria (see Hancock REW. Peptide antibiotics. Lancet 1997; 349: 418-22). Polymyxin exerts a bactericide effect with a mechanism similar than a detergent since the polycationic peptide ring is linked to the external membrane moving calcium and magnesium which stabilize LPS (15), generating changes in the permeability (see Verstovsek S, Maccubbin D L, Ehrke M J, Mihich E. Polymyxin B-mediated lysis of tumor cells. Int Arch Allergy Immunol. 1993;100(1):47-52) and the physico-chemical disruption of the membrane (see Hancock REW. Peptide antibiotics. Lancet 1997; 349: 418-22) allowing the pass of molecules including hydrophobic compounds and small proteins (15), generating the output of cell components (see Tam V H, Schilling A N, Vo G et al. Pharmacodynamics of polymyxin B against Pseudomonas aeruginosa. Antimicrob Agents Chemother 2005; 49: 3624-30) and the input of the polymyxin to the cell by an auto-promoted absorption pathway (see Hancock REW. Peptide antibiotics. Lancet 1997; 349: 418-22) generating cell death (see Alexandre Prehn Zavascki, Luciano Zubaran Goldani, Jian Li and Roger L. Nation. Polymyxin B for the treatment of multidrug-resistant pathogens:a critical review. Journal of Antimicrobial Chemotherapy (2007) 60, 1206-1215). In eukaryotes the mechanism of action is not known, however it is known that it would be totally dependent of the hydrophobic tail (non associated to the cycle) of these antibiotic (see Ferrari D, Pizzirani C, Gulinelli S, Callegari G, Chiozzi P, Idzko M, Panther E, Di Virgilio F. Modulation of P2X7 receptor functions by polymyxin B: crucial role of the hydrophobic tail of the antibiotic molecule. Br. J. Pharmacol. 2007 Feb; 150(4):445-54. Epub 2007).

CN1706454 discloses a medicine against cancer with synergistic features of bupleurum root extract and polymyxin being the weight ratio between both is 26.9-1724.1. bupleurum root is prepared by immersion in a solvent to obtain an extract separating the precipitated supernadant and drying thereof. The composition can effectively inhibit the cancer cell growth and requires a low dose of polymyxin, allowing a better drug resistance.

The current invention is related to a specific use of a known compound as complementary to conventional treatment against cancer, allowing the inhibition of the regulating response, depleting the regulating T lymphocytes by facilitating of P2X7 purinergic receptor activation, using the added effect of ATP and PMB in the depletion of Tregs CD4CD25 lymphocytes as a reinforce of immunotherapy against cancer. The ATP effect only can be related to several detrimental effects itself.

As follows it is accurately described the invention and so that the support of all features to be claimed later.

Ones of the aspects of the present invention is the PMB effect over lymphocyte subpopulations up to date only the added effect over transformed populations has been demonstrated without a clear function in reducing the regulating T responsive.

The treatment with PMB is able to induce the death of CD4+ CD25+ population, in vitro e in vivo, however it does not significantly affect to the lymphocyte T helper and T cytotoxic populations. The effect over the CD4+ CD25+ population depends on the dose with evaluated dose from 0.001 up to 1,000 μg/ml.

The current inventors also found that the depleting effect of PMB occurs at low concentrations, 100 times lower than the used in clinical and therapeutic procedures, and it is remarkable potentiated in vitro, as well as in vivo by the presence of low concentrations of NAD or ATP, non specific agonists to P2X7 receptor.

The CD4+ CD25+ lymphocyte depletion mediated by PMB, appears as being dependent in part of the activation of P2X7 receptor. Further it is specific to polymyxin B, since gramicidine other modulator described to P2X7 does not induce changes in the Treg population as well as niether in the conventional CD4 population.

The invention can be administered in way of dose units, intramuscularly, intradermally or intraperitoneally injected.

The use of an “effective dose” of “therapeutically effective dose” of PMB in animals in the treatment does not demonstrate the appearance of secondary effects over the health condition and regular behavior of animal model.

The use of PMB substantially improves the antitumor immunotherapy based on vaccines with tumor cell bodies increasing at 50% the effectiveness of the treatment in a murine melanoma model.

As previously discussed the depleting activity of PMB over the CD4+CD25 Treg cell population can be occupied in complementary way to the immunotherapy against cancer.

### SUMMARY

The objective of the present invention is the use of Polymyxin B as coadjuvant with ATP or NAD to the depletion of regulating T lymphocytes in mixtures of lymphocyte populations without significantly affecting the canonic lymphocyte populations.

Other objective of the invention is the proper in vitro conditions to achieve the effect of depletion of regulating T lymphocytes without significantly affect the remaining lymphocyte populations.

Even other objective of the present invention is the concentrations optimizing the effect of polymyxin B when used as complementary way in the treatment with ATP, in the depletion of regulating T lymphocytes.

Even more objective of the present invention is the mechanism by which polymyxin B exerts depletion on regulating T lymphocytes.

Even more objective of the present invention is the conditions in which the effect of polymyxin B is optimized when used as coadjuvant in the treatment with ATP to impede the tumor growth in mice to whom syngenic tumor cells has been injected.

### BRIEF DESCRIPTION OF DRAWINGS

FIGS. 1A and 1B depict the effect of PMB over CD8 y CD4 Treg cells presents in a total population of spleeny lymphocytes. PMB itself induces the Treg cell at concentrations equals or greater than 1 μg/ml, without affecting the CD8 population neither the CD4 population.

FIGS. 2A and 2B depict the gramicidine effect over the CD8 and CD4 Treg population presents in the total population of spleeny lymphocytes.

FIGS. 3A and 3B depict the effect of PMB over the effect induced by ATP. PMB at 1 μg/ml slightly modifies the effect induced by ATP, moving the curve forward down in parallel way to the normal control curve (without PMB), instead of PMB at 10 μg/ml produces a significant change in the response curve to ATP reflected in the depletion of Treg cells. This effect is observed also over the Foxp3+ population.

FIG. 4 depict a graphic where low dose of PMB and ATP are inhibited by non specific antagonists to P2X7, Each bar corresponds to the average of a minimum at four independent experiments to the treatment with PPADS 10 μM and BBG 100 nM together with the treatments PMB 1 μg/mL, ATP 60 μM, and the set of both over CD4CD25 populations. Bars correspond to the standard error of the analyzed data in each case.

FIG. 5 depict a graphic where BBG 100 nM only inhibits the effect induced by ATP 100 μM, but not PMB 10 μg/mL over CD4+ CD25+ population. Bars correspond to the standard error of the analyzed data in each case.

FIG. 6 depict a graphic where A740003, a specific antagonist to P2X7, inhibits only the induced effect by ATP 100 μM, but not of PMB 1, 10 neither 30 μg/mL over the CD4+ CD25+ population. Bars correspond to the standard error of the analyzed data in each case.

FIGS. 7A and 7B depict a dose of PMB+ATP inducing the death of systemic regulating lymphocytes T in an animal spleen treated reducing more than 50% between days 1 and 3 post treatment, at day 7 a partial recovering is produced. Other populations look slightly affected.

FIG. 8 depict PMB having a linear behavior in depletion of Tregs lymphocytes at a determined ATP dose, achieving a maximum depletion after the treatment with PMB 350 μg/Kg.

FIGS. 9A and 9B depict the tumor appearance incidence, control treatment, treatment with CA B16 alone and CA B16 plus ATP and PMB are depicted, each treatment was performed over a number of twelve individuals randomly without selecting a sex.

FIGS. 10A and 10B depict that PMB up to 400 μg total or ATP up to 50 μmoles does not induce the death of systemic regulating T lymphocytes in animal spleen at day 3 post challenge. Only a higher treatment of ATP 50 μg+PMB 40 μg induces a reduction of Treg in vivo. Other populations look slightly affected.

FIG. 11 depict the effect of PMB over Treg cells. PMB and ATP itself induces the death of Treg cells, the addition of both compounds slightly increases this reduction.

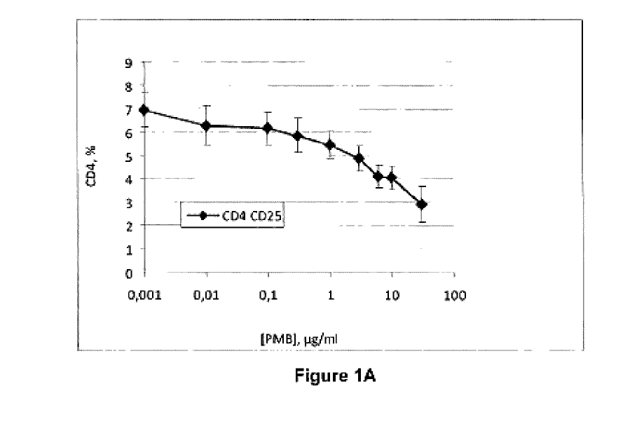

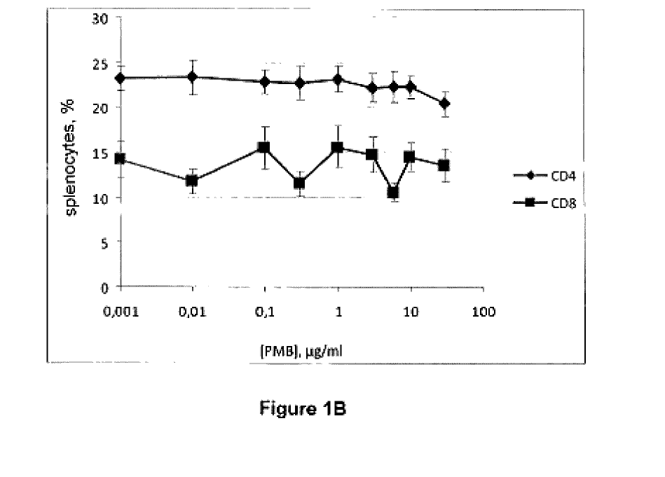

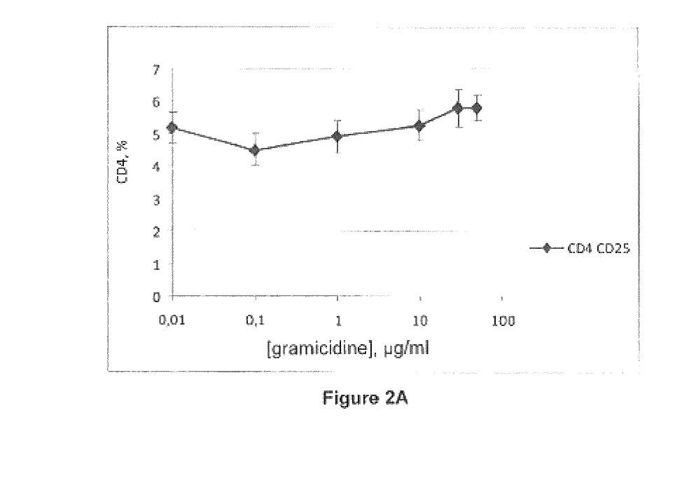

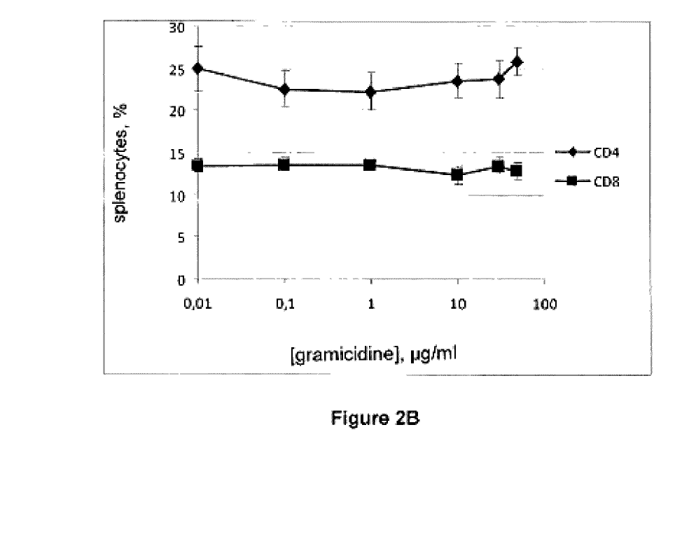

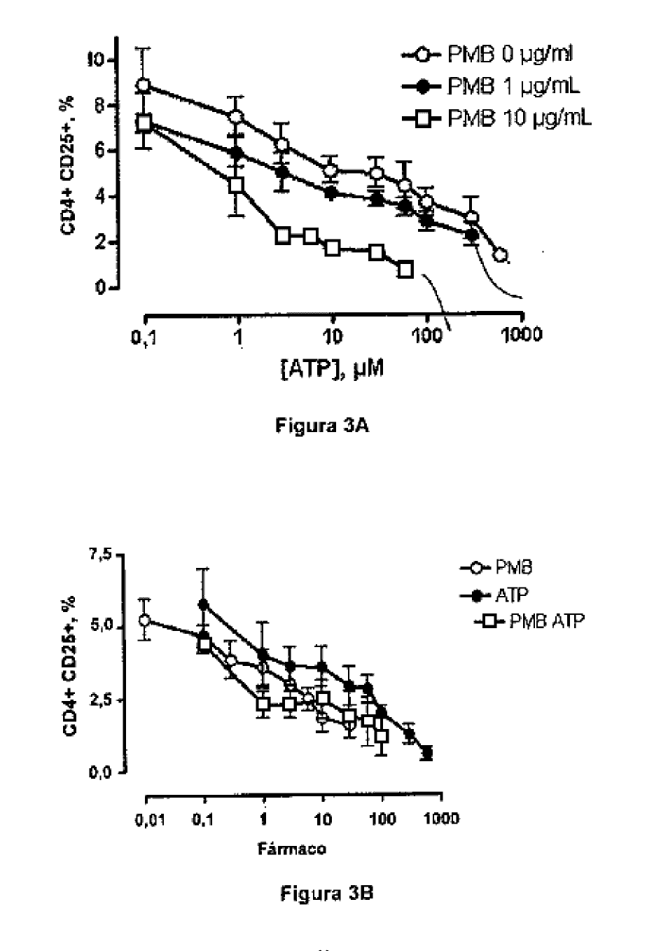

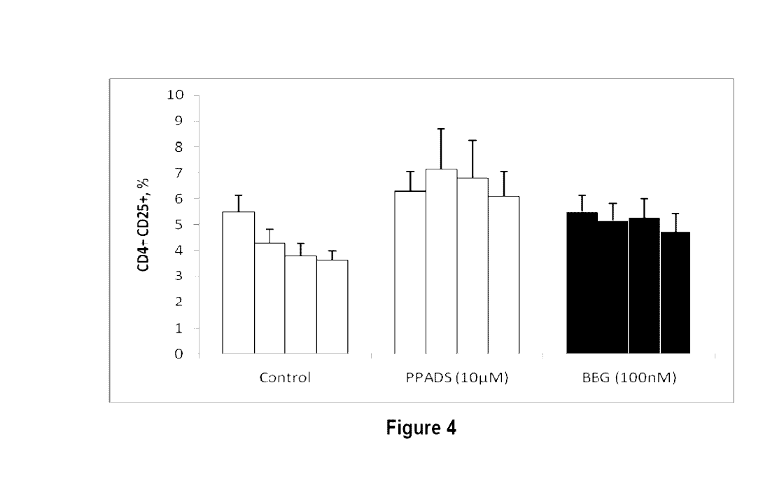

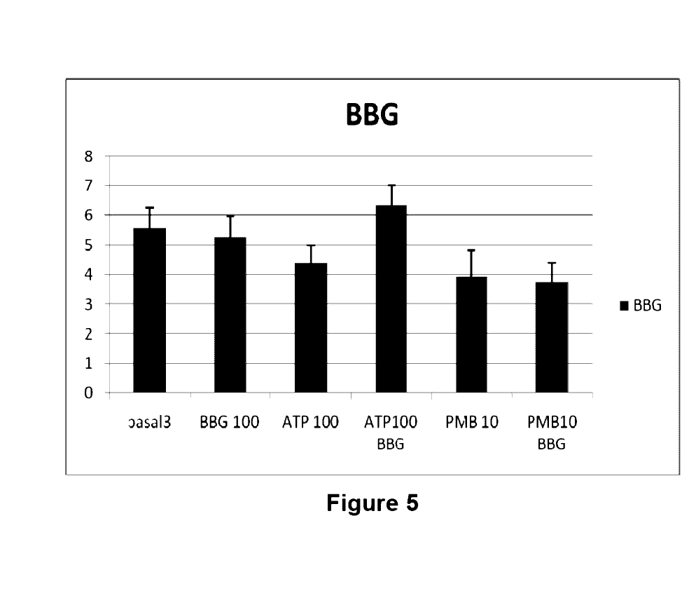

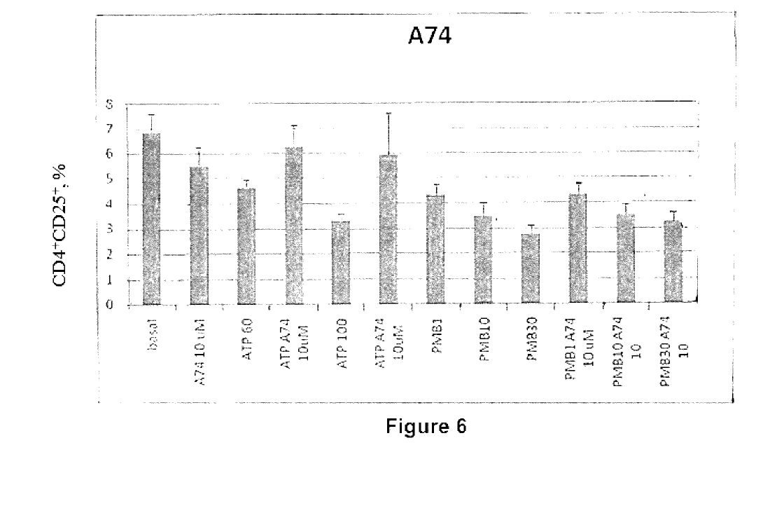

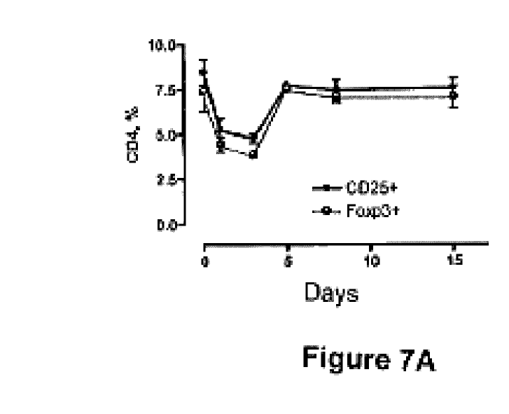

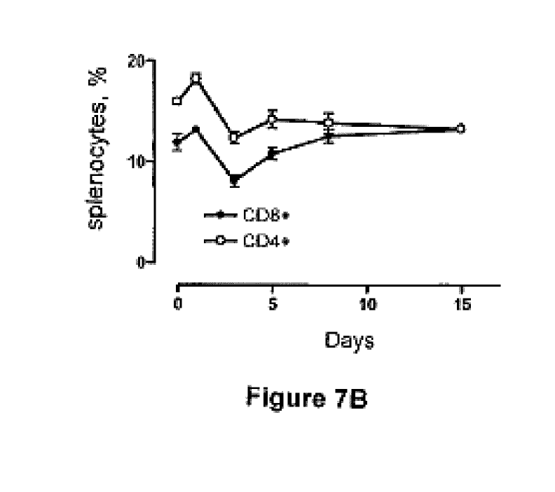

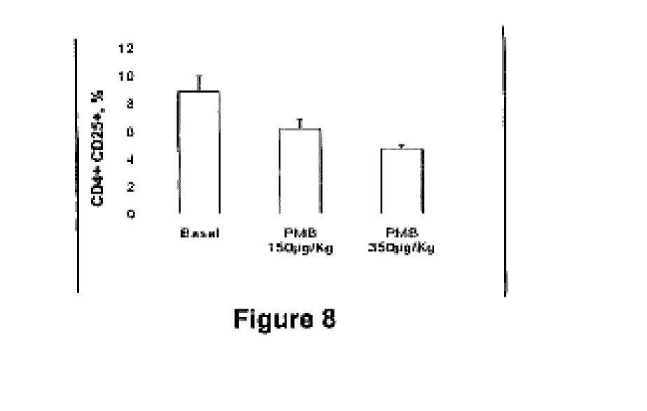

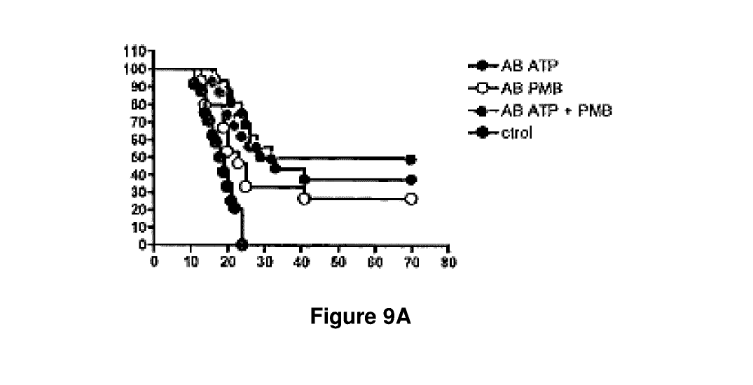

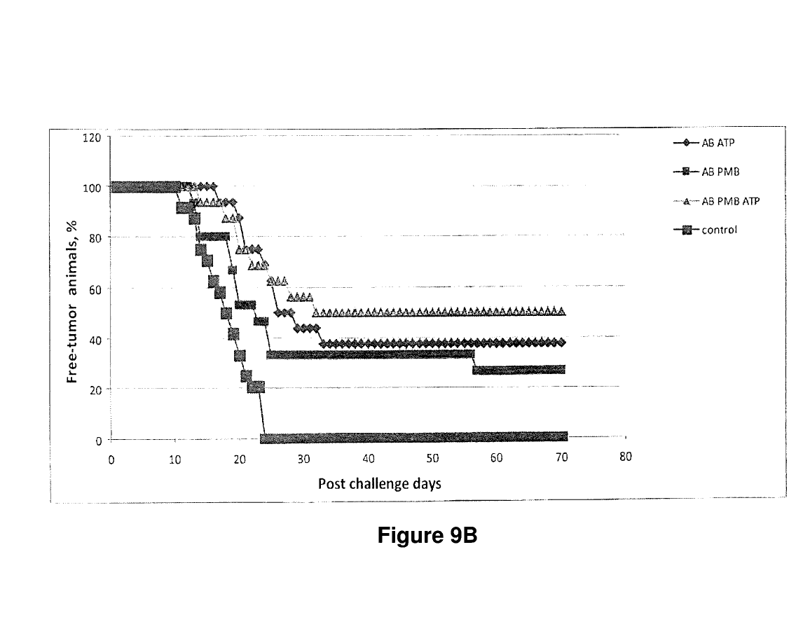

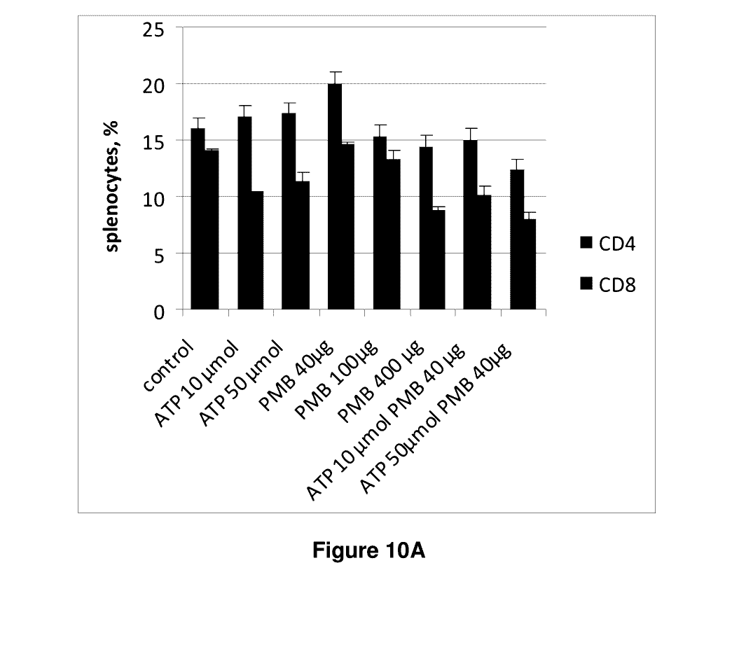

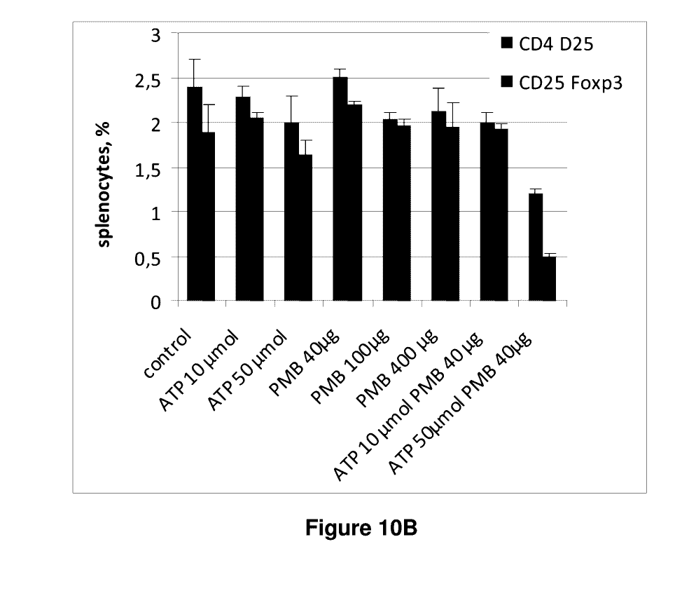

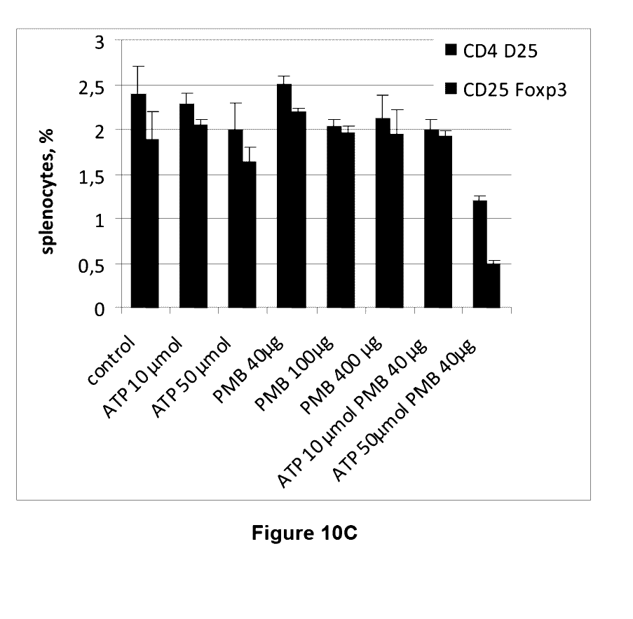

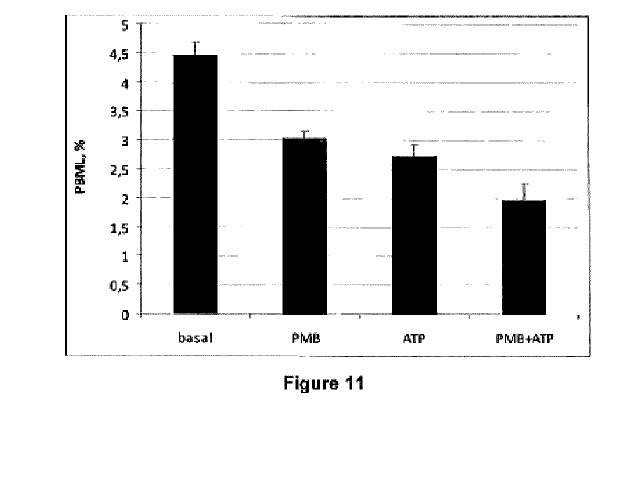

### DETAILED DESCRIPTION

To determine the in vitro effect of polymyxin and other receptor modulators: P2X7, spleen from mice b6 and/or balbc were extracted, and splenocytes were isolated using repeated centrifugation, eliminating red cells for treatment with a lysis buffer ACK. Generally, the experiments were performed in cells obtained from animals b6. The obtained splenocytes are cultured in RPMI Medium supplemented with antibiotics and serum at 10%. Cells remain in rest for 2 hours at a concentration of 1 million cells/ml, after the same are breeding and incubated with purinergic agonists dissolved in PBS (ATP 0.1 μM-1,000 μM and NAD 0.01-1,000 μM), modulators of PMB receptor (0.001 up to 1,000 μg/ml) and gramicidine (0.001 up to 100 mg/ml). In the case of antagonists, these are used at concentrations which have been described as being more specific to P2X7 than other receptors. These are dissolved in the solutions suggested by manufacturers and pre-incubated for 30 minutes before using the agonists or modulators.

Cells are cultured for different time under a RPMI culture environment supplemented with antibiotics (penicillin, streptomycin and fungizone) with serum fetal bovine serum at 10% in a culture stove at 37° C. and 5% CO₂, which will be named as standard conditions. To evaluate the effect of the treatment over regulating T lymphocytes, samples were taken for the treatments (independents) at 24, 48 and 72 hours post challenge. The recovered lymphocytes were centrifuged at 800 g and processed to be marked with antibodies against CD4, CD8, CD25. In some case they were made to waterproof to detection of foxp3 (all the blots were carried out by the use of a detection kit of mice regulating T lymphocytes of e-bioscience, according to the suggested by manufactures). Samples were analyzed in a cytometer of low and analyzed to different subpopulations to different evaluated times. The results obtained herein allow determining that PMB in vitro under the described conditions, selectively depleting to splenocyte regulating T lymphocytes.

To evaluate the complementary PMB activity in antitumor therapy, first is determined the effect of this over the in vivo regulating T population. To this, animals having 6-15 weeks and different sex were subcutaneously injected with a solution containing PMB alone (between 4 to 400 μg total), ATP (between 0,4 a 40 mg total) or mixture thereof, in ratios which are not affected therebetween, the CD4CD25 foxp3 population. Animals were dead are different post-injection time, spleen was extracted, erythrocytes eliminated and T lymphocytes present evaluated. The results obtained herein allow determining that PMB injected depleting regulating T lymphocytes under a transient way.

To evaluate the complementary PMB activity in antitumor therapy, cells B16 were cultured under standard conditions in a culture DMEM medium with fetal bovine serum at 10% in a culture stove at 37° C. and 5% CO₂. Cells were cultures and submitted to dead induced by deprivation of culture medium for 96 hours, into a culture stove. Cell bodies obtained were collected in sterility conditions and stored at a concentration estimated considering 1 million initial cells/ml. These bodies were prepared together with PMB and ATP inducing a reduction of T reg lymphocytes and injected 3 times each 7 days. After 7 days, once ended the immunization protocol, animals were challenged with 20.000 cells b16 alive obtained from exponential growing culture. Tumor growth was evaluated by hand after the first week daily and measure with a vernier calliper. The results obtained herein allow determining that PMB in vivo plus ATP and with cell bodies act in a prophylactic way increasing the number of animals free of tumor after the challenge with alive b16 cells.

The results obtained allow determining that polymyxin B, independent from the use as antibiotic to Gram Negative bacteria, is useful in the treatment of cancer acting as deplector of regulating T lymphocytes together with in vitro ATP or NAD. The administering methodologies as well as the concentrations are subsequently described. The use of this drug can be a mechanism less toxic to eliminating regulating T lymphocytes, which is associated to a immunotherapy treatment can induce a immune responsive resolute against cancer. This drug associated to ATP, allow together with these nucleotides reducing the regulating T lymphocyte population and can be occupied with any other immunotherapy requiring depletion of regulating T lymphocytes.

### EXAMPLES

Example 1

### Effect of PMB over Helper T Lymphocyte Populations and Treg (Mouse)

Splenocytes 2×10⁶ cells/ml obtained from spleen of mice C57/BL6 (6 weeks old) were cultured by 1 hour in plates 24 wells at 37° C., 5% CO₂. Lately, cells were treated with PMB (Sigma-Aldrich, St Louis, Mont., USA) to different times and concentrations (0-24-48 and 72 hours, concentrations from 0.01 a 1,000 μg/ml). The treatments were kept for 24, 48 and 72 h, in which cells were kept into RPMI-1640 medium supplemented at 37° C. 5% CO₂. After the treatments cells were harvested in buffer IF (PBS 2% SFB) could without dragging the adherent population. The obtained cells were blocked at 4° C. for 30 min in buffer IF. To determine the CD4+, CD8+ y CD4+ CD25+ FOXP3+ lymphocyte populations, about 10⁶ cells were incubated with Anti-CD8 Ly-2 (BDpharmigen, Mississauga, Ontario, USA) 4° C. for 30 min with constant stirring and CD4+ T lymphocytes and Treg were identified with the kit of marking cells Treg #3 (eBioscience, San Diego, Calif., USA) as the manufacturer protocol. After the incubation cells were re-suspended in buffer IF and analyzed by flow cytometry in the equipment FACSCantoll (Beckett Dickinson, Mississauga, Ontario, USA). The same experiments above described were repeated evaluating the effect of gramicidine, ATP and ATP in presence of two PMB concentrations.

This example establishes that the PMB effect is specific and determine the required conditions to depleting the CD4+ CD25+ FOXP3+ populations.

### Example 2

### The PMB Effect on Treg Cells is Mediated in Part by P2X7R

Splenocytes were treated with PMB (Sigma-Aldrich, St Louis, Mont., USA) to different times and concentrations in presence of two modulators negative of the P2X7, PPADS activity and specific antagonist of P2X7 Brilliant Blue G (BBG) (see Brilliant Blue G Selectively Blocks ATP-Gated Rat P2X7 receptors. JIANG, 2000. 12). Cells were kept for 24 hrs in conditions of cell culture. Subsequently cells were recollected, blotted and analyzed by flow cytometry.

From this example it is possible to conclude that the PMB effect over the depletion of regulating T lymphocytes partially depends on the P2X7 receptor activation.

### Example 3

### PMB Induces the in vivo Treg Lymphocyte Depletion

Subcutaneous injections were performed in mice C57/BL6 of 6 weeks old by different days each drug alone or with PMB 150 μg/Kg and 350 μg/Kg in combination with a fixed ATP dose (1 g/Kg); which corresponds to described doses as no damaging to human beings. Mice were dead, the spleen was extracted and then marking with α-CD4, αCD25, α-CD8 and α-Foxp3 antibodies were made. (*=p<0.05).

So that, PMB can potentiate the depletion of Treg lymphocytes after 72 hrs of in vivo exposition.

### Example 4

### Evaluation of PMB and ATP in Antitumor Immune Therapy

Mice from 6 to 8 weeks old were treated with apoptotic bodies (CA) derived from Melanome murine cells B16 (2×10⁴), with CA B16 plus ATP 500 μM, CA B16 plus PMB 10 mg/mL and with mixtures if the above three, the control was performed with the sterile immunization carrier PBS 1X.

Immunizations were performed by subcutaneous injection in the right flank in a final volume of 50 μL, at day 0, 7 and 21 of treatment. Day 0 marks the starting of the treatments. In day 28 post injection a tumor challenge was made. To that 10⁴ tumor cells alive were injected in the left flank of the individual in a volume of 50 μL in sterile PBS 1X.

Mice were daily observed recording activity, weight and aspect according to parameters described by Morton y Griffiths, 1985 (see Morton D B, Griffiths P H. Guidelines on the recognition of pain, distress and discomfort in experimental animals and an hypothesis for assessment. Vet Rec. 1985 Apr 20;116(16):431-6). The tumor appearance was recorded and followed up to a tumor volume of 0.6 cm³, in which animals were euthanized by cervical dislocation.

The use of cell bodies confers protector immunity in 20% of animals at day 20 after inoculation of tumor cells. Immunotherapy treatment with consistent support of PMB addition only confers a protection of 60% of the challenged animals. The addition of PMB and ATP allow achieving a protection of 100% animals at day 20. The evaluation at day 60 post inoculation gives protection percentages of 0%, 20% and 50% respectively.

From this example, it is concluded that the treatment of cell bodies plus ATP or PMB, induces prophylaxis against the development of tumor b16 in mice and that the sum of both compounds improves this prophylaxis.

### Example 5

### PMB Induces the in vivo Depletion of Human Treg Lymphocytes

Subcutaneous injections were made in mice C57/BL6 of 6 weeks old with different concentrations of PMB, ATP or mixing concentrations of both. Mice were dead, the spleen extracted and subsequently markings were made with α-CD4, αCD25, α-CD8 and α-Foxp3 antibodies. (*=p<0.5).

### Example 6

### The PMB Effect over Human Treg Lymphocyte Populations

PBML 2×10⁶ cells/ml obtained from peripheral blood extracted by centrifugation in gradient was cultured under standard conditions. Subsequently cells were treated with PMB (Sigma-Aldrich, St Louis, Mont., USA). The Treatments were kept for 24 h, in which cell remain in RPMI-1640 medium supplemented at 37° C. 5% CO₂. After the treatments cells were harvested in buffer IF (PBS 2% SFB) cold. The obtained cells were blocked at 4° C. for 30 min in buffer IF. To determine the CD4+, CD8+ and CD4+ CD25+ FOXP3+ lymphocyte populations. After incubation cells were resuspended in buffer IF and analyzed by flow cytometry in the FACSCantoll equipment (Beckett Dickinson, Mississauga, Ontario, USA).

1. A method for treating cancer comprising the steps of administering to a subject in need thereof, a combination including an effective amount of polymyxin B in combination with an effective amount of ATP, wherein the combination is effective in treating a tumor or cancer in the subject.

2. The method of claim 1, wherein the subject is a mammal.

3. The method of claim 1, wherein the combination is administered together.

4. The method of claim 1, wherein the cancer is melanoma.

5. A method of treating cancer comprising the steps of:
isolating a lymphoid tissue including spleen tissue, blood or a combination thereof comprising splenocytes from a subject suffering from cancer;
cultivating the splenocytes in vitro;
submitting the splenocytes to a medium comprising a combination of from 0.1 μM to 1,000 μM ATP and from 0.1 μg/ml to 1,000 μg/ml of polymyxin B, wherein the combination is effective in depleting the number of CD4⁺CD25⁺ Tregs in the total splenocyte population; and
administering back to the subject Treg-depleted splenocytes, including dead Treg cells.
